(19) United States
(12) Patent Application Publication
(10) Pub. No.: US 2025/0342948 A1
(43) Pub. Date: Nov. 6, 2025

(54) DEVICE AND METHOD FOR THE INTERACTIVE RELEASE OF AN OPERATING MODE

(71) Applicants: FRESENIUS MEDICAL CARE DEUTSCHLAND GMBH, Bad Homburg (DE); FRESENIUS MEDICAL CARE MAGYARORSZÁG EGÉSZSÉGÜGYI KFT., Budapest (HU); FRESENIUS MEDICAL CARE GMBH, Bad Homburg (DE)

(72) Inventors: Thiemo DALDRUP, Bad Homburg (DE); Christopher HENNEKEN, Bad Homburg (DE); Michael CHRIST, Bad Homburg (DE); Thomas POHL, Bad Homburg (DE); Alexander WAGNER, Bad Homburg (DE); Ferenc VERES, Bad Homburg (DE)

(73) Assignees: FRESENIUS MEDICAL CARE DEUTSCHLAND GMBH, Bad Homburg (DE); FRESENIUS MEDICAL CARE MAGYARORSZÁG EGÉSZSÉGÜGYI KFT., Budapest (HU); FRESENIUS MEDICAL CARE GMBH, Bad Homburg (DE)

(21) Appl. No.: 18/876,202

(22) PCT Filed: Jun. 16, 2023
(86) PCT No.: PCT/EP2023/066256

§ 371 Date: Dec. 18, 2024

(30) Foreign Application Priority Data
Jun. 24, 2022 (DE) 10 2022 115 865.9

(51) Int. Cl. G16H 40/40

(52) U.S. Cl. CPC G16H 40/40

(57) ABSTRACT

The present invention relates to a method for automatically, dynamically approving an operating mode of a system, wherein the device has a user interface and a control unit, which is programmed to automatically determine the most recent measured value available from a database with regard to a plurality of parameters recorded on the basis of an input made by means of the user interface as required for an operating mode to be approved, to compare the determined measured value in each case with a setpoint value and/or a tolerance range surrounding this and to output an indication of the comparison result and its criticality by means of the user interface for each determined measured value, preferably directly adjacent to the measured value, wherein the output by means of the user interface is in the form of an overview of the parameters recorded as required, which enables a user to centrally approve or reject the operating mode to be approved with regard to the plurality of parameters recorded as required.

The present invention relates to a device and a method for interactively approving an operating mode of a system or at least one component thereof, in particular in the field of medical technology and/or water technology in a medical context; for example for producing dialysis water, ultrapure water, etc.

In clinics, before operating treatment devices, such as dialysis facilities, to ensure patient safety, it must first be ensured that the water produced for this purpose meets the required quality regulations. For this purpose, the water technology used, for example a water treatment system, must function flawlessly. Before operating treatment devices, the associated water technology and/or water quality must therefore always be tested and approved before it can be operated; for example, to provide dialysis water. Here, the inspection and approval of the water technology system and/or the water supplied by the water technology system should be carried out as directly as possible before the desired use, in order to provide little room for sources of error occurring again.

In larger clinics, such testing and approval is not traditionally possible. In most cases, the water technology system is checked manually, which is why the checking users sometimes have to walk very long distances to check the components of the water technology system, e.g. a reverse osmosis system, a chlorine sensor or a filtration unit, which are sometimes located in different rooms of a clinic, or to read the corresponding sensors.

In practice, this conventional procedure results in long time delays, which mean that when the last measured value is determined, for example, the first measured value is no longer up-to-date. This puts patient safety at risk.

In addition, valuable time is traditionally lost even in the event of a detected malfunction, as the user must first independently determine and then contact the responsible contact person; for example, an attending physician or a service technician.

By the time the final approval is given, for example because the attending physician considers a recorded deviation in water temperature to be uncritical, further sources of error may thus have occurred.

Furthermore, the delays make the planning of the usually already hectic daily routine at the clinic more difficult, since it can only be decided quite late whether a planned treatment is to be carried out, changed, postponed or cancelled altogether.

Against this background, the object is to mitigate or even completely eliminate the disadvantages of the prior art. In particular, the object of the present invention is to increase patient safety and the user-convenience of the approval process.

This object is achieved by the subject-matters of the independent claims. Further developments of the invention are the subject-matter of the sub-claims.

Consequently, a device for interactively approving an operating mode of a system, in particular a water technology system, or at least a component thereof, is provided, wherein the device has a user interface and a control unit, which is programmed to, in each case, automatically determine the most recent measured value available from a database with regard to a number of parameters recorded on the basis of an input made by means of the user interface as required for an operating mode to be approved, to compare the determined measured value in each case with a setpoint value and/or a tolerance range surrounding this and to output an indication of the comparison result and/or its criticality by means of the user interface for each determined measured value, preferably directly adjacent to the measured value, wherein the output by means of the user interface is in the form of an overview of the parameters recorded as required, which enables a user to centrally approve or reject the operating mode to be approved with regard to the number of parameters recorded as required.

The number of parameters can comprise one or several parameters. The parameters can reflect a water quality and/or a state of the system or at least a component thereof.

For the approval of the operating mode of the water technology system, required parameters can be parameters relating to the water quality and/or relating to the state and/or status of the water technology system. Water quality parameters can be parameters of the input water, the delivered pure water or delivered dialysis water and/or parameters that occur in the system between the input stage and the output stage. These can be parameters related to the water pre-treatment, for example. Water pre-treatment usually has at least one physical filter, an activated carbon filter and a softener stage. A parameter can be measured on or between the filter stages. One parameter that changes significantly over the stage of the activated carbon filter, for example, is the chlorine content as a quality parameter of the water, because an activated carbon filter removes chlorine from water flowing through it. So, a useful measurement can be the chlorine content after flowing through the activated carbon filter. Water quality parameters can also be understood as general properties of the water, such as water temperature.

Preferably, the term “interactive approval” is to be understood in the sense that a device according to the invention interacts with a user to perform an operation of a device; for example, an operation of a water technology system for producing dialysis water. For example, the approval process is performed jointly by the device and one or more human users and/or contact persons, wherein the device assists and/or guides the human users and/or contact persons through the approval process.

For example, in a device according to the invention, a user makes the input that an operation of a water technology system for the production of dialysis water is to be approved.

Based on this input, the device preferably automatically records which parameters are to be taken into account during the approval and are thus required. For example, it can be stored in a database that it is necessary to check the temperature of the produced water and its chlorine content upon approval of a water technology system for the production of dialysis water. Instead of the formulation of required parameters, the formulation of relevant or critical parameters can also be chosen.

The parameters stored in the database as to be required may be based on user input or, for example, may have been recorded on the basis of regulatory requirements currently in force at a particular location.

For example, the user may have stored in the database that when approving a water technology system for the production of dialysis water, the parameters required for approving this operating mode are the temperature of the produced water and its chlorine content. Furthermore, the user may have stored in the database that when approving a water technology system for the production of pure water, the parameters required for the approval of this operating mode are the hardness of the produced water and its chlorine content.

These examples are not to be understood as limiting, but serve merely to illustrate that the parameters stored in the database as required for approval can be set by a user.

Alternatively or additionally, it can be provided that the device, after the input by the user that an operation of a water technology system for the production of dialysis water is to be approved, automatically accesses the current regulatory provisions applicable at a location of the water technology system and automatically extracts from these the parameters required for the approval.

For example, due to the applicable legal provisions in a country X at a time X, it may be a requirement that the parameters required for approving this operating mode are the temperature of the produced water and its chlorine content. In this example, the device thus records or extracts the parameters temperature and chlorine content, which are required for approval.

However, in country Y, at the same time X, it may be a regulation, that when a water technology system is approved for the production of dialysis water, the parameters required for the approval of this operating mode are the hardness of the water produced and its chlorine content. In this example, the device thus records or extracts the hardness and chlorine content parameters required for approval.

These examples are not meant to be limiting, but merely to illustrate how parameters can be recorded as required for approval.

Basically, only one parameter may be required for an approval, but also several parameters may be required for an approval.

For each parameter required for the approval, a device according to the invention preferably in each case determines the most recent measured value; for example, the most recent available measured value of the water temperature or the water hardness.

The fact that the device automatically determines the most recent measured value available for each recorded parameter, for example by using a database, preferably eliminates the need for the user to manually read the measured values at various locations, usually on foot, and manually feed them into the device.

Preferably, the available measured values are collected in real time or approximately in real time and thus always reflect the current state of the system.

The device directly and/or indirectly and wirelessly and/or by wire communicates with the system and/or a database in which the measured values are stored.

Preferably, each determined measured value of each parameter required for the approval is compared with a setpoint value.

A system with an operating mode approved by means of the present device or method preferably comprises at least one of the following components, but possibly also several or all of them: water pre-treatment with at least one physical filter, a softener and an activated carbon filter, a reverse osmosis system, sensors and a monitoring device, one or more gateways that transmit data from one of the aforementioned components to a server or a Cloud; alternatively, the reverse osmosis system, water pre-treatment and/or sensor device can themselves be capable of data communication via a network or have an integrated gateway, a ring line for transporting water, to which medical devices, in particular dialysis machines, can be connected.

The respective setpoint value and/or the tolerance range surrounding it can either be set by the user, result from the technical provisions of the system or component thereof and/or result from legal or regulatory provisions or medical specifications, for example of the attending physician.

Alternatively or additionally to the setpoint value, there can also be a setpoint range.

Preferably, the respective measured value is displayed to the user as well as an indication of the comparison result of the measured value with the setpoint value, e.g. “value increased” or “value increased, but still within the tolerance range”.

The indication of the comparison result can also take the form that measured values below the threshold value are displayed in a reduced font size relative to a standard size and measured values above the threshold value are displayed in an enlarged font size relative to a standard size.

The indication of the criticality of a deviation of a measured value from a setpoint value and/or a tolerance range surrounding it can be carried out, for example, as a color code, as a numerical scale or also by adjusting the size of the measured value (the larger the deviation, the larger the measured value is displayed). The indication of criticality may include the display of a symbol, e.g. a flag or star, in the vicinity of a particular measured value.

For example, a color code in the manner of a traffic light can be provided, in which measured values that correspond to the setpoint value or a setpoint range are marked in green or are not marked at all, measured values that fall within a tolerance range are marked, for example, by yellow symbols or flags, and measured values that fall outside the tolerance range are marked, for example, by red symbols or flags.

However, these explanations are only exemplary in nature and the indication of criticality can take on any form as long as the extent to which a measured value deviates from a target value is indicated.

Preferably, the output is in the form of an overview of the parameters and/or the associated measured values recorded as necessary for the approval in such a way that a user can grasp all parameters and/or associated measured values and indications of their criticality at a glance and can approve the operating mode to be approved or reject the approval with preferably only one click.

Additionally, it can be provided that the user is also shown the parameters and/or measured values that are not mandatory for approval. For example, a device according to the invention can be configured such that the user can select, by means of an input, which parameters and/or measured values are to be displayed in the overview in addition to the parameters recorded as required for approval.

Preferably, the output or overview contains a summarized indication or a status display of the state of the system established by the device, for example, by providing a color code in the overview in the manner of a traffic light, by means of which the state of the entire system is assessed as being correct (green), slightly deviating from the target state or in the tolerance range (yellow) or significantly deviating (red). In this way, the user preferably already receives a summary of the overall state of the system at a glance. If, for example, the state of the entire system has been directly assessed as green, the user preferably does not have to look at individual measured values, but can directly approve the operating mode.

According to one embodiment, the device is further configured to automatically document an approval or rejection thereof made by the user, preferably including a time stamp, name, position and/or authorization of the user, recorded parameters, determined measured values, operating mode to be approved and indication of the criticality of individual deviations and/or the summarized indication of the state of the system created by the device. The documentation is preferably carried out in a digital logbook taking into account the currently applicable local legal and regulatory requirements, to which the device preferably automatically adapts the documentation.

According to one embodiment, the control unit is programmed to automatically detect, on the basis of an input regarding the operating mode to be approved, the plurality (or number, since in principle only one parameter may be required for enabling) of parameters required (or also critical and/or relevant) for this operating mode, preferably with recourse to information regarding the type, a current or planned operating mode, a current or planned maintenance mode and/or the location of the system or the component thereof and/or the regulatory provisions currently applicable at this location.

For example, the selected operating mode to be approved may be “Providing dialysis water”. The device then determines, preferably automatically, the components of the system required to that end, e.g. the reverse osmosis system, as well as the parameters relevant for checking the function of these components, e.g. the pressure in a particular line. Alternatively, the parameters required to approve an operating mode can also be specified by the user or another authorized person.

According to one embodiment, the control unit is programmed to automatically determine at least one responsible contact person from a database in the event of a user rejection of the approval of the operating mode, for at least one but in principle also for each measured value that has been identified as deviating from the associated setpoint value and/or tolerance range, and preferably to establish a notification to the contact person(s), which enables them to approve the operating mode to be approved with reference to the respective measured value or to reject the approval.

The notification(s) is/are only sent once the user has agreed to the sending. Preferably, at least one measured value is provided in a notification that has been identified as deviating from the associated setpoint value and/or tolerance range.

In other words, according to one embodiment, a two-stage or multiple-stage approval process is provided.

Preferably, the device determines the respective contact person automatically.

If, for example, a deviating pressure value has been recorded in a water pipe, the responsible contact person may be a service technician. If, for example, a temperature deviation of the water to be prepared has been recorded in a water pipe, the responsible contact person may therefore be the attending physician. Preferably, the contact persons and their assignment to certain measured values and/or parameters are stored in a database.

The responsible contact person can also vary with the criticality of a deviation or its size, so that, for example, an approval of a treatment has to be approved by an assistant physician despite minor deviations, but an approval must be made by a head physician in the case of major deviations.

It can further be provided that a device according to the invention detects an authorization and/or a position (e.g. service technician, doctor, etc.) of the user and then determines with regard to which parameters the user is authorized to approve and/or with regard to which parameters an approval by a contact person is required; in other words, a two-step approval process.

For example, if the device records that the water temperature and chlorine content parameters of the water are required for an approval process, and further records that the user is a service technician, the device may, for example, only enable the user to approve despite a deviating chlorine content reading (since it is assumed that a service technician can judge the correct operation of a dechlorinator), but may require notification of a contact person (e.g. a doctor) during approval despite a deviating water temperature reading, as a doctor can be expected to better judge whether a deviating water temperature is dangerous to a patient.

In other words, a device according to the invention can be designed to record an authorization and/or a position of a user and/or contact person, and to limit the approval authority of the user and/or contact person to the parameters and/or measured values corresponding to the authorization and/or position. Which parameters and/or measured values correspond to which authorization and/or position is preferably stored in a database.

The notification to a contact person can be established wirelessly or wired and the respective contact person can be contacted via email, messenger services, push notifications, SMS, social media or by other means.

According to one embodiment, the control unit is programmed to, in the event of a user rejection to approve the operating mode, determine a responsible contact person from a database and preferably to establish a notification to the contact person only if the deviation of the measured value from the setpoint exceeds a certain threshold value of criticality and/or the measured value lies outside a tolerance range surrounding the setpoint value and/or if the measured value belongs to a recorded parameter required for the approval of the operating mode to be approved.

This provides the advantage that no two-stage approval process is initiated in the case of minor deviations that are assessed as non-critical by the device. This reduces the burden on contact persons and further increases the user-friendliness of the approval process. For example, a doctor stored as a contact person for the parameter temperature does not receive a notification for every minor deviation of the temperature (e.g. 37.1° C. instead of 37° C.), but only if the deviation exceeds certain levels of insignificance and the measured value is outside a tolerance range (e.g. is 37.5° C., where the set value is 37° C. and the tolerance range is +/−0.4° C.).

For example, it can also be provided that if the criticality of the comparison result is displayed, for example, in the manner of a traffic light, a contact person is only notified in the event of comparison results of a measured value to a setpoint value classified in the “red” category.

For example, if a user rejects the approval of operation of the water technology device for providing dialysis water because a deviation of the measured value from the setpoint value classified in the “red” category has been recorded for one parameter, e.g. water temperature, and a deviation of the measured value from the setpoint value classified in the “yellow” category has been recorded for another parameter, e.g. water hardness, it can be provided that the device does not determine a contact person and establish a notification for both of the parameters water temperature and water hardness, but only for the contact person regarding the water temperature, since only for this parameter is there a deviation classified in the “red” category.

It can be provided that contrary to such a default setting of the device, a user can select that the contact person should nevertheless also be notified with regard to the water hardness.

If a contact person is responsible for several parameters, the device can be designed to combine the notifications regarding the several parameters in a common overview, preferably ordering or sorting the notifications according to urgency; for example, based on the criticality of an associated comparison result (e.g. deviations classified in the “red” category are communicated first or come at the top of a list).

According to one embodiment, the control unit is programmed to automatically establish a content, in particular a text content, for at least one, a plurality or also each notification to the respective responsible contact person.

According to one embodiment, the control unit is programmed to automatically insert into at least one, a plurality of or also each notification to the respective responsible contact person an option to view via electronic remote access for recording the deviating measured value and/or its criticality and/or an associated state report of the system or the component thereof, preferably in the form of a link.

Alternatively or additionally, the deviating measured value and/or an indication of the criticality of the deviation and/or an associated state report of the system of the component can be included in a notification.

This gives the contact person the possibility to gain his/her own impression of the state of the system as well as the deviating measured value without the need to consult the user.

Preferably, the link is a deep link, i.e. preferably an address for access via a digital data link that leads directly to the deviating measured value and/or the indication of the criticality of the deviation and/or the associated state report of the system or component. For example, access authorizations or an authorization and/or identity of a user or contact person can also be encoded in the link.

According to one embodiment, the control unit is programmed to approve or start the operating mode in the event of a user rejection to approve the operating mode immediately after approving one or more deviating measured values by the respective responsible contact person, without requiring the user to approve this again.

This configuration also provides the option of accelerating the approval process, as no consultation of the contact person with the user and a subsequent approval by the user is required.

According to one embodiment, the control unit is programmed to, in an event in which a contact person is responsible for several parameters or measured values, carry out the notification in the form of an overview of the deviating measured values, which enables the contact person to centrally approve the operating mode to be approved with regard to the plurality of deviating measured values or to reject this being approved.

This preferably enables a contact person to record the relevant measured values and/or parameters at a glance and to approve or reject the operating mode in a centralized manner, preferably with just one click.

For example, a service technician receives an overview that the pressure in a certain line is 3.8 bar instead of the setpoint value of 4 bar and a flow rate in another line is 898 l instead of the setpoint value of 890 l.

In this case, the overview contains these values, for example, which the service technician can preferably tick off or mark as harmless by means of a button. The service technician can then approve or reject the operating mode with a click on a corresponding button without having to approve or reject these separately for each measured value or parameter. The step of ticking off or marking the individual values as harmless is optional.

According to one embodiment, the control unit is programmed to automatically determine the most recent measured value available immediately before a planned start of the operating mode to be approved from a database with regard to the plurality of parameters recorded as critical and/or to execute the entire approval process immediately before a planned start of the operating mode to be approved.

This reflects the particularities of the field of medical technology or water technology in the medical sector, where there is a need for the water produced or used to always and immediately meet the highest requirements. Thus, monitoring of the function of systems or their components and their approval as quickly as possible, repeated at high frequency, is required.

Preferably, a device according to the invention is configured to determine the most recent available measured value and/or to execute the entire approval process in a period of time between 1 minute and 30 minutes, preferably between 1 minute and 20 minutes, in particular between 5 minutes and 20 minutes before the planned start of the operating mode to be approved.

A device according to the invention can be, for example, a laptop, computer, tablet or other electronic device that is used, for example, in a control room or in a control center of a clinic.

Examples of parameters recorded include a conductivity, a chlorine content, a flow volume, a flow rate, a total volume of water produced, a water hardness, a temperature, a pressure, a membrane permeability, an age of a component, e.g. a filter cartridge, a maintenance status, e.g. hot disinfection overdue. However, this list is only exemplary and other parameters can be considered alternatively or additionally.

Another aspect of the invention relates to a computer-implemented method for interactively approving an operating mode of a system, in particular of a water technology system, or at least a component thereof, wherein the method is preferably carried out by means of a device according to the invention, with the steps of:
automatically determining from a database the most recent measured value available for a number of parameters recorded as required for approving an operating mode to be approved, based on input made by means of the user interface,
comparing the determined measured value in each case with a setpoint value and/or a tolerance range surrounding this, and
outputting, by means of the user interface for each determined measured value, preferably immediately adjacent to the measured value, an indication of the comparison result and its criticality, wherein
   the output by means of the user interface is in the form of an overview of the parameters recorded as critical, which enables a user to centrally approve or reject the operating mode to be approved with regard to the plurality of parameters recorded as critical.

The above-mentioned explanations with respect to a device according to the invention are also applicable to a method according to the invention, even if not all features are explicitly repeated in order to avoid redundancies.

According to one embodiment, the plurality of parameters critical for this operating mode is automatically detected on the basis of an input regarding the operating mode to be approved, wherein preferably there is recourse to information regarding the type, a current or planned operating mode, a current or planned maintenance mode and/or the location of the system or of the component thereof and/or the regulatory provisions currently applicable at this location.

According to one embodiment, in the event of a user rejection to approve the operating mode, for each measured value that has been identified as deviating from the associated setpoint value and/or a setpoint range and/or a tolerance range, at least one responsible contact person is automatically determined from a database by means of the control unit and preferably a notification is established to them, which enables the contact person to approve the operating mode to be approved with reference to the respective measured value or to reject the approval.

Preferably, in this case a notification contains the option for digital remote access; for example, in the form of a link or deep link. A deep link is preferably an address for remote access via a digital data connection that preferably leads directly to the approval request or the approval.

For example, a button that a contact person can click on can be provided in a notification. When the button is operated, a link opens that enables the contact person to remotely act on the device and make an approval. Here, the contact person preferably does not have to log in separately or confirm an identity, because the identity, position, role, authorization and/or approval authority of the contact person are preferably coded in the deep link.

The deep link can lead from one level of interaction with the contact person to a lower-level instance of a web-based application.

Different contact persons can have different approval authorizations that are preferably stored in a database to which the device has access. The approval authorizations can be related to the role or position of a contact person (doctor, service technician, etc.).

It can be provided that several contact persons receive notifications, but only one of these contact persons has approval authority and the other contact persons receive the notification for information only. For example, it can be provided that only a doctor can give his/her approval, but service technicians nevertheless also receive notifications in order to be informed about any deviating measured values and, if necessary, to carry out appropriate work on the system.

According to one embodiment, in the event of a user rejection to approve the operating mode, a responsible contact person is determined for a measured value from a database and preferably a notification is established to the contact person only if the measured value lies outside a tolerance range surrounding the setpoint value and/or if the measured value belongs to a recorded parameter required for the approval of the operating mode to be approved and/or the deviation of the measured value from the setpoint value exceeds the criticality by a certain threshold value.

It can be provided that for at least one, a plurality or each notification or for a number of notifications to the respective responsible contact person, a content, in particular a text content, is automatically created.

Furthermore, an option to view the deviating measured value and/or an associated status report of the installation or the component thereof, preferably in the form of a link is automatically inserted into each notification or a number of notifications to the respective responsible contact person by means of electronic remote access.

In the event of a user rejection to approve the operating mode, the operating mode can be approved or started immediately after approving one or more deviating measured values by the respective responsible contact person, without requiring the user to approve this again. Alternatively, an (additional) approval may also be required from the user.

In an event in which a contact person is responsible for several parameters or measured values, the notification of this contact person can be made in the form of an overview of the deviating measured values, which enables the contact person to approve or reject the operating mode to be approved with regard to the plurality of deviating measured values in a centralized manner, preferably with only one action, such as a click.

According to one embodiment, automatically determining the most recent measured value available immediately before a planned start of the operating mode to be approved from a database with regard to the plurality of parameters recorded as critical and/or the entire approval process is carried out immediately before a planned start of the operating mode to be approved.

Preferably, the measured values can be determined and/or the entire approval process can be executed in a period of time between 1 minute and 30 minutes, preferably between 1 minute and 20 minutes, in particular between 5 minutes and 20 minutes before the planned start of the operating mode to be approved.

Preferably, a method according to the invention would comprise a step of automatic or self-actuating documentation. For example, it can be provided to automatically document an approval or rejection made by the user, preferably including a time stamp, name, position and/or authorization of the user, recorded parameters, determined measured values, operating mode to be approved and indication of the criticality of individual deviations and/or the summarized indication of the state of the system created by the device. The documentation is preferably carried out in a digital logbook taking into account the currently applicable local legal and regulatory requirements, to which the device preferably automatically adapts the documentation.

Another aspect of the invention relates to a data carrier, which contains machine-readable instructions, enabling a device to perform a method according to the invention. Furthermore, such a data carrier can also be used for retrofitting an existing device in order to retrofit it within the meaning of the present invention. A data carrier within the meaning of this application preferably also comprises a data storage space in the Cloud.

Further features, effects and advantages of the present application result from the following description of exemplary embodiments of the invention with reference to the Figures. In the Figures:

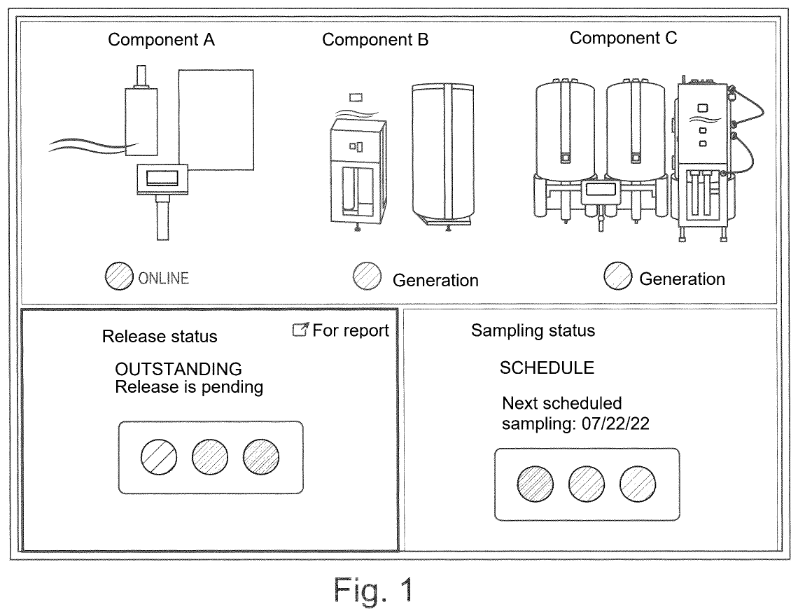

FIG. 1 shows an exemplary view of a summarized indication of the status of a system before the approval;

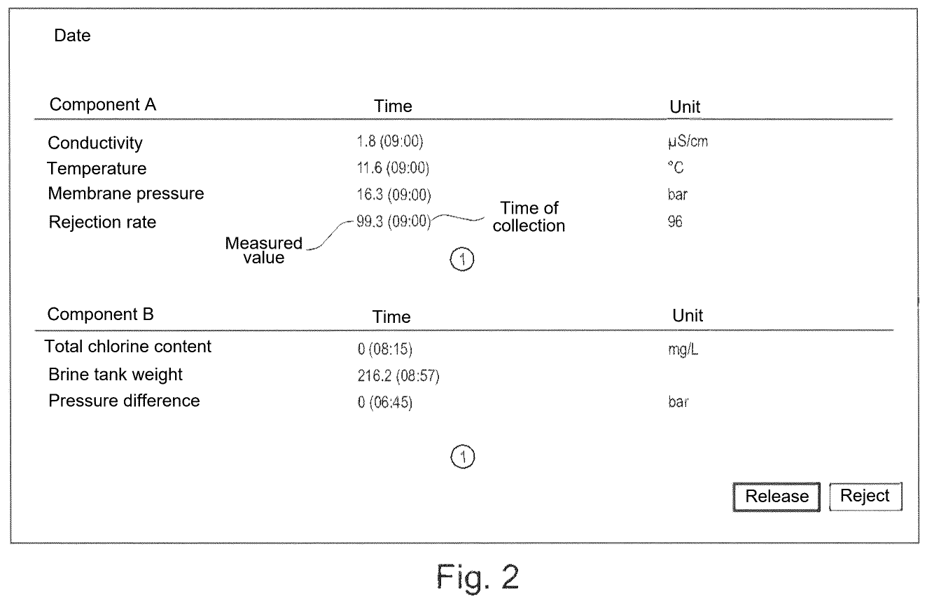

FIG. 2 shows an exemplary view of an overview of recorded parameters and their measured values as well as a button to approve or reject the approval;

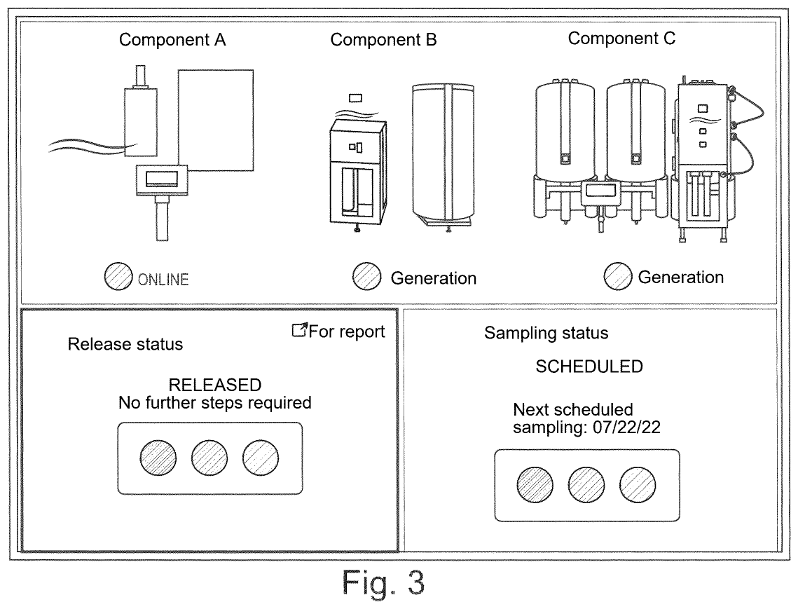

FIG. 3 shows an exemplary view of a summarized indication of the condition of a system after the approval;

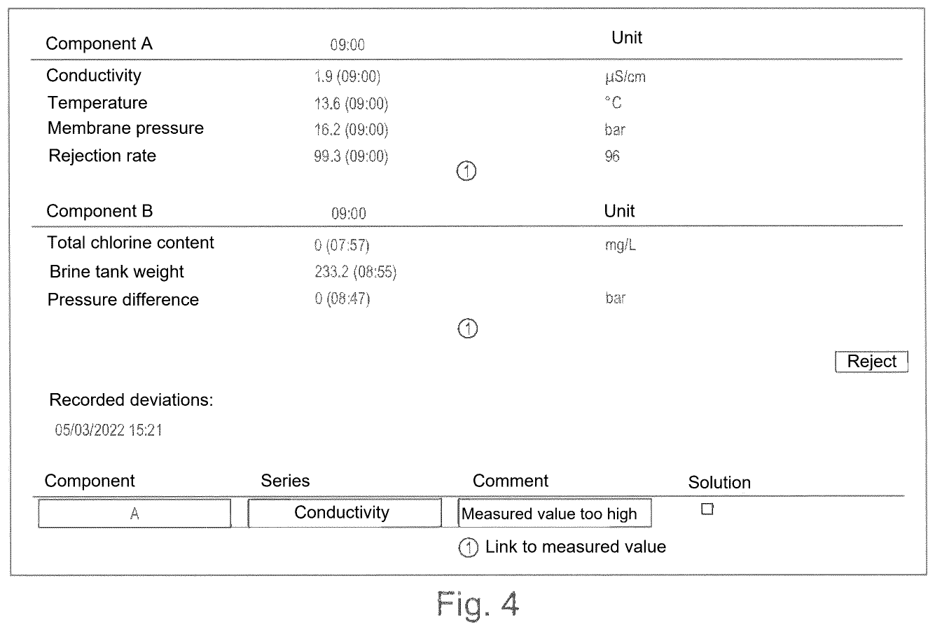

FIG. 4 shows an exemplary view of an overview displayed to a user, by means of which a recorded deviation can be transmitted to a contact person for further approval;

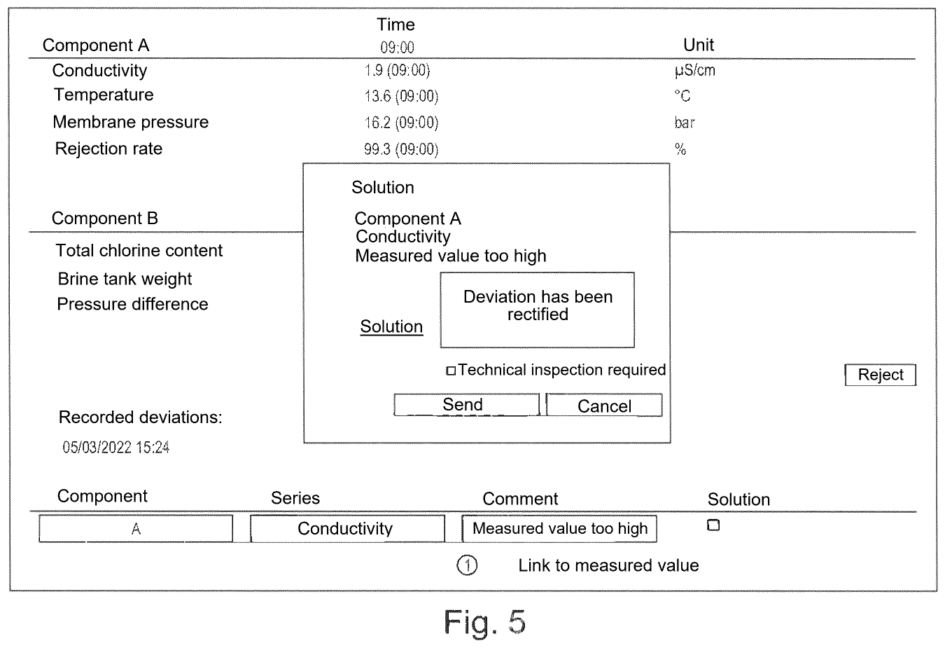

FIG. 5 shows an exemplary view of an overview displayed to a contact person, by means of which they can comment on a recorded deviation and approve or reject it;

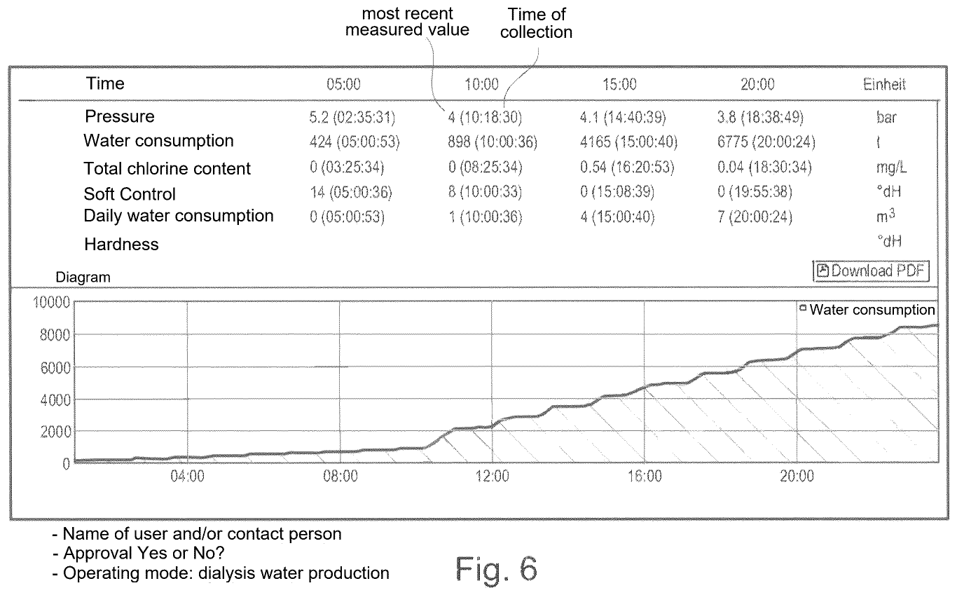

FIG. 6 shows an exemplary view of documentation from which the determination of the most recent measured values results;

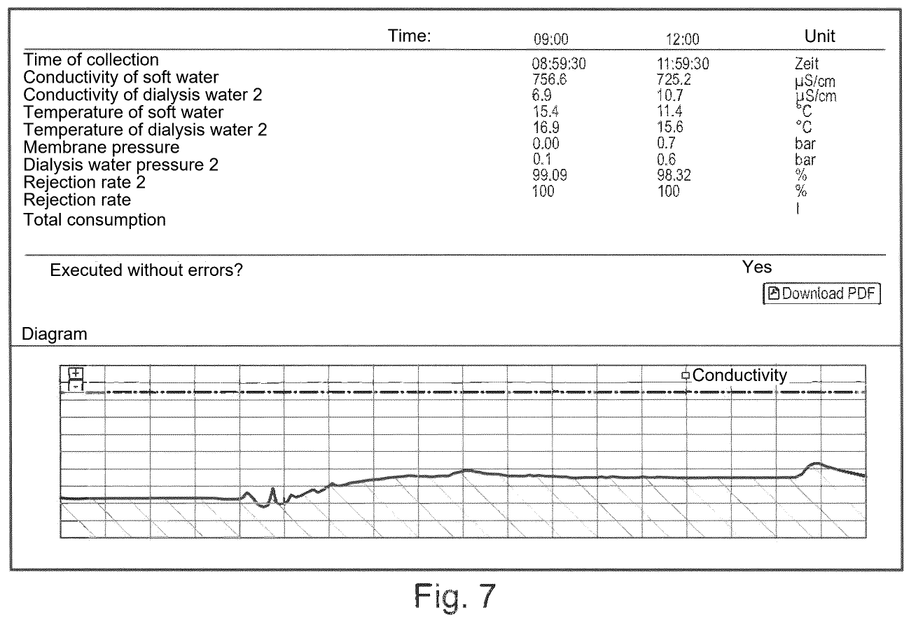

FIG. 7 shows an exemplary view of a documentation, which reflects the recorded measured values in the course of a day; and

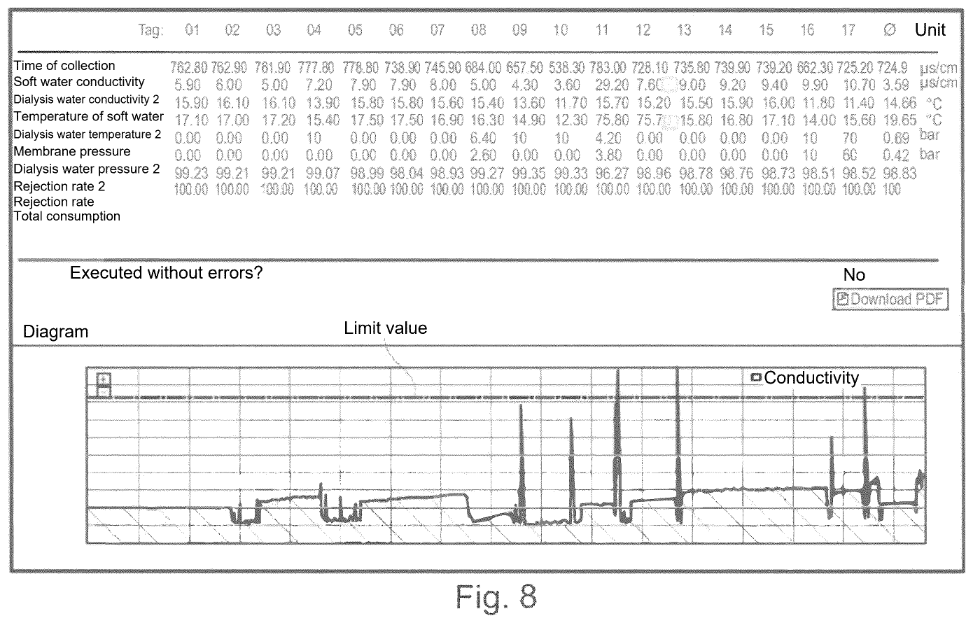

FIG. 8 shows an exemplary view of a documentation, which reflects the recorded measured values over 17 days, wherein on day 12 two errors were recorded, the criticality of which is displayed.

As shown in FIG. 1 in an exemplary manner, according to one embodiment, a summary of the state of a system having the components A to C is displayed to the user by means of a device according to the invention. In this example, the operating state of the system, in particular of the components A to C for generating dialysis water, has not yet been approved; the approval is therefore pending and the displayed traffic light is red. The sampling status is according to plan, e.g. no sampling was omitted and the associated traffic light is therefore green.

FIG. 2 shows an exemplary view of an overview of recorded parameters and their measured values as well as a button to approve or reject the approval. Such a view is shown to a user of the device, for example.

For an approval at 9:00 a.m., the most recent measured values of the parameters (in this example conductivity, temperature, membrane pressure, etc.) are determined. For example, the most recent measured value of conductivity is a measured value of 1.8 μS/cm, which was taken at 9:00 a.m. The most recent measured value of the total chlorine content is a measured value of 0 mg/l, which was taken at 8:15 a.m.

In this example, no deviations or malfunctions were recorded. Using the “Approve” and “Reject” buttons, the user can approve or reject the operating mode with just one click. Once approved, the system starts to generate dialysis water.

It can be provided that the approval is only possible once a measured value has been determined for all parameters marked as critical or particularly important.

In the view in FIG. 2, a particularly important or critical parameter, in this example the conductivity, has been highlighted by a display with increased line width (bold). The view of the user is thus drawn directly to the most important parameter values.

FIG. 3 shows an exemplary view of a summarized indication of the condition of a system after the approval. In this example, the operating state of the system, in particular of the components A to C for generating dialysis water, has been approved; the approval has therefore been carried out and the displayed traffic light is green. The sampling status is also according to plan, e.g. no sampling was omitted and the associated traffic light is therefore green.

FIG. 4 illustrates a case in which a deviation of a measured value from a setpoint has been recorded and a contact person is to be contacted. FIG. 4 shows an exemplary view of an overview displayed to a user, by means of which a recorded deviation can be transmitted to a contact person for further approval.

As not all measured values are within the target range, the user rejects an approval using the “Reject” button. The device thus automatically generates a summary of the deviation to the contact person. Using a command field, the user can transmit additional information to the contact person. In this example, the conductivity measured value is too high.

FIG. 5 shows an exemplary view of an overview displayed to a contact person, by means of which they can comment on a recorded deviation and approve or reject it. In this example, the contact person is a service technician, for example. For example, after the notification, the service technician resolved the problem and conductivity is back in the target range. After resolving the problem, the service technician can, as shown in FIG. 5, enter, for example using a link in the notification, which can lead to an approval screen and/or the measured value, that the problem has been resolved and approve the operating mode. In this example, the service technician enters that the problem has been resolved by means of a comment function. The service technician can then send this feedback and preferably approve the operating mode or cancel the process.

FIG. 6 shows an exemplary view of documentation from which the determination of the most recent or closest measured values results. For example, a pressure value of 4 bar was assigned to the measurement at 10 a.m., which was taken at 10:18:30 a.m.

Preferably, the device has an automatic assignment; for example, in the form of a closest match algorithm, which assigns the closest measured values in each case to scheduled recording periods or points in time. The measured values can be before or after the scheduled recording periods or points in time.

For example, as part of the automatic documentation, release reports are automatically generated by the device, which, as shown in FIG. 6, for example, contain the measured values assigned to the various recording points in time, the corresponding parameters, such as pressure, water consumption, total chlorine content, etc., the name and position of the user and the contact person(s), as well as an indication of whether an approval has been established or not.

FIG. 7 shows an exemplary view of a documentation, which reflects the recorded measured values for the recording points in time 9:00 a.m. and 12:00 p.m. Furthermore, in this view an indication is provided that no malfunctions have been recorded.

An evaluation and/or documentation over longer time intervals is also possible. FIG. 8 thus shows an exemplary view of documentation, which reflects the recorded measured values over 17 days, wherein on day 12 two errors were recorded, the criticality of which is displayed. Thus, the conductivity value in column 2 of day 12 is highlighted with a yellow flag and the temperature value in column 4 of day 12 is highlighted with a red flag. Furthermore, in this view an indication is provided that a malfunction has been recorded.

In the view in FIG. 8, the progress of the conductivity measured value is shown as a diagram. The limit value is also drawn in the diagram; if the limit value is exceeded, a measured value is marked with a yellow flag. In this way, a viewer can get an overview of the size of the exceedance of the limit value at a glance.

In addition, the person skilled in the art understands that if an element is provided in the singular with the article “a” or “one”, this is not to be understood as “exactly one”, but is to be understood in the sense of “at least one”, wherein the disclosure is not limited to exactly one of these elements.

In addition, it is obvious for the person skilled in the art that if certain elements or features of the invention are disclosed in a certain combination or in connection with a certain embodiment, these elements or features of the invention can also be isolated or claimed in other combinations or in connection with another embodiment.

All features disclosed in connection with the method according to the invention may equally relate to a device according to the invention and vice versa, even if, for the sake of brevity, not all of these features or combinations thereof are expressly disclosed.

1. A device for interactively approving an operating mode of a system, or at least a component thereof, wherein the device has a user interface and a control unit that is programmed to automatically read or load into a memory from a database in each case the most recent available measured value at least with regard to a number of parameters recorded as being necessary for approving the operating mode,
compare the determined measured value in each case with a setpoint value and/or a tolerance range surrounding this and to output an indication of the comparison result and/or its criticality by means of the user interface for each determined measured value, wherein
the output by means of the user interface is in the form of an overview of the measured values of the parameters recorded as required, which enables a user to centrally approve or reject the operating mode to be approved with regard to the determined measured values of the parameters required for approving the operating mode.

2. The device according to claim 1, wherein the control unit is programmed to automatically detect, on the basis of an input regarding the operating mode to be approved and/or regarding a type of the system or of the at least one component thereof, the number of parameters required for approving this operating mode, wherein there is recourse to information regarding the type, a current or planned operating mode, a current or planned maintenance mode and/or the location of the system or of the component thereof and/or the regulatory provisions currently applicable at this location.

3. The device according to claim 1, wherein the control unit is programmed to, in the event of a user rejection to approve the operating mode, automatically determine from a database at least one responsible contact person for each measured value identified as deviating from the associated setpoint value, and to establish and, in the event of confirmation of the sending by the user, to send an electronic notification to the contact person, wherein the notification enables the contact person to approve the operating mode to be approved or to reject the approval by means of electronic remote access to the device with reference to the respective measured value.

4. The device according to claim 3, wherein the control unit is programmed to, in the event of a user rejection to approve the operating mode, determine at least one responsible contact person for a measured value from a database and to establish a notification to the at least one contact person only if the measured value lies outside a tolerance range surrounding the setpoint value and/or if the measured value belongs to a recorded parameter required for the approval of the operating mode to be approved.

5. The device according to claim 3, wherein the control unit is programmed to automatically create a content for at least one notification to at least one responsible contact person.

6. The device according to claim 3, wherein the control unit is programmed to automatically insert into at least one notification to at least one responsible contact person an option to view the deviating measured value and/or an associated status report of the installation or the component thereof in the form of a link.

7. The device according to claim 1, wherein the control unit is programmed to approve or start the operating mode in the event of a user rejection to approve the operating mode immediately after approving one or more deviating measured values by the respective responsible contact person, without requiring the user to approve this again.

8. The device according to claim 1, wherein the control unit is programmed to, in an event in which a contact person is responsible for several parameters or measured values, to carry out the notification in the form of an overview of the deviating measured values, which enables the contact person to centrally approve the operating mode to be approved or to reject the approval with regard to the plurality of deviating measured values.

9. The device according to claim 1, wherein the control unit is programmed to automatically determine the most recent measured value available immediately before a planned start of the operating mode to be approved from a database with regard to the plurality of parameters recorded as required for the approval and/or to execute the entire approval process immediately before a planned start of the operating mode to be approved.

10. A computer-implemented method for interactively approving an operating mode of a system, or at least a component thereof, wherein the method is carried out by means of a device according to claim 1, with the steps of:
automatically determining the most recent measured value available for a number of parameters recorded as required for an operating mode to be approved from a database by means of a control unit,
comparing each determined measured value in each case with a setpoint value and/or a tolerance range surrounding this value by means of the control unit, and
outputting, by means of a user interface for each determined measured value, immediately adjacent to the measured value, an indication of the comparison result and/or its criticality, wherein
the output by means of the user interface is in the form of an overview of the determined measured values of the parameters recorded as required for the approval, which enables a user to centrally approve or reject the operating mode to be approved with regard to the number of determined parameters.

11. The method according to claim 10, wherein on the basis of an input regarding the operating mode to be approved and/or regarding a type of the system or of the at least one component thereof, at least one parameter required for the approval of this operating mode is automatically detected by means of the control unit, the number of parameters required for approving this operating mode is automatically detected by means of the control unit, f, wherein there is recourse to information regarding the type, a current or planned operating mode, a current or planned maintenance mode and/or the location of the system or of the component thereof and/or the regulatory provisions currently applicable at this location.

12. The method according to claim 10, wherein, in the event of a user rejection to approve the operating mode, for each measured value identified as deviating from the associated setpoint value and/or lying outside of the tolerance range, at least one responsible contact person is automatically determined from a database by means of the control unit and an electronic notification is issued to the contact person and, in the event of confirmation of the sending by the user, and sent to the contact person, wherein the notification enables the contact person to approve the operating mode to be approved or to reject the approval by means of electronic remote access to the device with reference to the respective measured value.

13. The method according to claim 12, wherein, in the event of a user rejection to approve the operating mode, at least one responsible contact person is determined from a database and a notification for the contact person is established, only if the measured value lies outside a tolerance range surrounding the setpoint value or the permissible range and/or if the measured value belongs to a recorded parameter required for the approval of the operating mode to be approved.

14. The method according to claim 12, wherein a content, which comprises at least one measured value, is automatically established for each notification to the respective responsible contact person.

15. The method according to claim 12, wherein an option to view the deviating measured value and/or an associated status report of the system or the component thereof, in the form of a link is automatically inserted into at least one notification to at least one responsible contact person.

16. The method according to claim 10, wherein the operating mode is immediately approved or started in the event of a user rejection to approve the operating mode after approving one or more deviating measured values by the respective responsible contact person by means of electronic remote access to the device, without requiring the user to approve this again.

17. The method according to claim 10, wherein, in an event in which a contact person is responsible for several parameters or measured values, the notification is made in the form of an overview of the deviating measured values, which enables the contact person to centrally approve the operating mode to be approved with regard to the several parameters or measured values or to reject this being approved.

18. The method according to claim 10, wherein the most recent measured value available is automatically determined immediately before a planned start of the operating mode to be approved from a database with regard to the number of the parameters recorded as required for the approval and/or the entire approval process takes place immediately before a planned start of the operating mode to be approved.

19. A data carrier, which contains machine-readable instructions, enabling a device to perform the method according to claim 10.
